(19) United States
(12) Patent Application Publication
(10) Pub. No.: US 2024/0392248 A1
(43) Pub. Date: Nov. 28, 2024

(54) HUMAN BONE MARROW-DERIVED MESENCHYMAL STEM CELL SHEETS AND METHODS FOR THEIR PRODUCTION

(71) Applicants: UNIVERSITY OF UTAH RESEARCH FOUNDATION, Salt Lake City, UT (US); SCM LIFESCIENCE INC., Incheon (KR)

(72) Inventors: Teruo OKANO, Salt Lake City, UT (US); David W. GRAINGER, Salt Lake City, UT (US); Masatoshi OKA, Salt Lake City, UT (US); Kyungsook KIM, Salt Lake City, UT (US); Sumako KAMEISHI, Salt Lake City, UT (US); Sun UK SONG, Incheon (KR); Celia M. DUNN, Salt Lake City, UT (US)

(21) Appl. No.: 18/697,153

(22) PCT Filed: Sep. 30, 2022
(86) PCT No.: PCT/US2022/045435

§ 371 Date: Mar. 29, 2024

Related U.S. Application Data
(60) Provisional application No. 63/278,136, filed on Nov. 11, 2021.
(60) Provisional application No. 63/251,144, filed on Oct. 1, 2021.

(51) Int. Cl. C12N 5/0775; A61L 27/38

(52) U.S. Cl. CPC C12N 5/0663; A61L 27/3834; A61L 2430/26; C12N 2513/00; C12N 2533/30; C12N 2539/10

(57) ABSTRACT

The disclosure provides a human bone marrow-derived mesenchymal stem cell (hBMSC) sheet comprising one or more layers of human hBMSCs, wherein the cell sheet is prepared from a human clonal bone marrow-derived mesenchymal stem cell line generated from a single cell. The disclosure also provides methods for producing human bone marrow-derived mesenchymal stem cell sheets comprising culturing hBMSCs in culture solution on a temperature-responsive polymer which has been coated onto a substrate surface of a cell culture support, wherein the temperature-responsive polymer has a lower critical solution temperature in water of 0-80° C.; adjusting the temperature of the culture solution to below the lower critical solution temperature, whereby the substrate surface is made hydrophilic and adhesion of the cell sheet to the surface is weakened; and detaching the cell sheet from the culture support. The cell sheet may be treated with interferon gamma (IFN-γ) or basic fibroblast growth factor (bFGF).

### RELATED APPLICATIONS

This application claims priority to U.S. Provisional Patent Application No. 63/251,144 filed on Oct. 1, 2021, and U.S. Provisional Patent Application No. 63/278,136 filed on Nov. 11, 2021, the contents of each of which are incorporated by reference herein in their entirety.

### BACKGROUND OF THE INVENTION

Mesenchymal stem cells (MSCs) are pluripotent somatic stem cells that can differentiate into osteoblasts, chondrocytes, nerve cells, skeletal muscle cells, vascular endothelial cells, and myocardial cells (Reyes et al., 2002, J. Clin. Invest. 109; 337-346; Toma et al., 2002, Circulation 105, 93-98; Wang et al., 2000, J. Thorac. Cardiovasc. Surg. 120, 999-1005; Jiang et al., 2002, Nature 41S, 41-49). Therapeutic properties of MSCs are proposed to derive from their intrinsic ability to 1) differentiate into multiple and distinct cell lineages, 2) produce an array of soluble bioactive factors central to cell maintenance, survival and proliferation, 3) modulate host immune responses, and 4) migrate as recruited to sites of injury to mitigate damage and promote healing (Squillaro et al., 2016, Cell Transplant, 25(5), 829-848). In particular, human bone marrow-derived mesenchymal stem cells (hBMSCs) are an allogeneic cell source of particular interest due to the secretion of multiple paracrine factors such as immunomodulatory factors (e.g., Interleukin-10: IL-10, prostaglandin E2: PGE-2), anti-fibrotic factors (e.g., hepatocyte growth factor: HGF, Bone morphogenetic protein 7: BMP-7), and angiogenic factors (e.g., Vascular endothelial growth factor: VEGF, basic fibroblast growth factor: bFGF). Thus hBMSCs have great potential for a variety of therapeutic uses.

### SUMMARY OF THE INVENTION

In certain aspects the disclosure relates to a human bone marrow-derived mesenchymal stem cell sheet comprising one or more layers of human bone marrow-derived mesenchymal stem cells (hBMSCs), wherein the cell sheet is prepared from a human clonal bone marrow-derived mesenchymal stem cell line generated from a single cell. In certain embodiments, the hBMSCs in the cell sheet are confluent. In certain embodiments, the human clonal bone marrow-derived mesenchymal stem cell line has been frozen before preparation of the cell sheet. In certain embodiments, the human clonal bone marrow-derived mesenchymal stem cell line has not been frozen before preparation of the cell sheet. In certain embodiments, the cell sheet consists essentially of hBMSCs. In certain embodiments, at least 90% of cells in the cell sheet are hBMSCs. In certain embodiments, the hBMSCs in the cell sheet express one or more cytokines selected from the group consisting of human growth factor (HGF), vascular endothelial growth factor (VEGF), Fibroblast Growth Factor 2 (FGF2), interleukin-10 (IL-10), Indoleamine 2,3-dioxygenase (IDO), and Prostaglandin E2 (PGE2). In certain embodiments, expression of the one or more cytokines in the cell sheet is increased relative to a suspension of hBMSCs containing an equivalent number of cells. In certain embodiments, initial seeded cell density of the human clonal bone marrow-derived mesenchymal stem cell line in a cell culture support used to prepare the cell sheet is from 4.5×10⁴ to 3.4×10⁵ cells/cm².

In certain aspects the disclosure relates to a composition comprising an hBMSC cell sheet as described herein and a polymer-coated culture support that is removable from the cell sheet.

In certain aspects the disclosure relates to a method for producing a human bone marrow-derived mesenchymal stem cell sheet comprising one or more layers of confluent human bone marrow-derived mesenchymal stem cells (hBMSCs), the method comprising:
a) culturing hBMSCs in culture solution on a temperature-responsive polymer which has been coated onto a substrate surface of a cell culture support, wherein the hBMSCs in culture solution are a human clonal bone marrow-derived mesenchymal stem cell line generated from a single cell, and wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

In certain embodiments, the hBMSCs in culture solution have been frozen before the culturing step (a). In certain embodiments, the hBMSCs in culture solution have not been frozen before the culturing step (a). In certain embodiments, the culturing step (a) comprises adding hBMSCs to the culture solution at an initial cell seeding density from 4.5×10⁴ to 3.4×10⁵ cells/cm².

In certain embodiments, the method further comprises culturing the hBMSCs through multiple subcultures prior to the culturing step (a). In certain embodiments, 2 to 10 subcultures of the hBMSCs are performed prior to the culturing step (a). In certain embodiments, the hBMSCs are cultured in the culture solution on the temperature-responsive polymer for at least 1 day before the adjusting step (b). In certain embodiments, the hBMSCs are cultured in the culture solution on the temperature-responsive polymer for 1 to 3 days. In certain embodiments, the adjusting step (b) is performed when the hBMSCs in culture solution are confluent.

In certain aspects the disclosure relates to an hBMSC sheet produced by the methods described herein.

In certain aspects the disclosure relates to a method of transplanting a cell sheet to a subject comprising applying the cell sheet of any one of claims 1 to 10 or 20 to a tissue of a subject. In certain embodiments, the tissue is kidney tissue. In certain embodiments, the subject has received a kidney transplant. In certain embodiments, the subject has an acute kidney injury. In certain embodiments, the subject has kidney tubule injury. In certain embodiments, applying the cell sheet to the kidney tissue results in migration of cells from the cell sheet into parenchyma tissue of the kidney. In certain embodiments, applying the cell sheet to the kidney tissue results in increased levels in the kidney of one or more cytokines selected from the group consisting of human growth factor (HGF), vascular endothelial growth factor (VEGF), Fibroblast Growth Factor 2 (FGF2), interleukin-10 (IL-10), Indoleamine 2,3-dioxygenase (IDO), and Prostaglandin E2 (PGE2), relative to a kidney that is not contacted with the cell sheet.

In certain aspects the disclosure relates to a method of suppressing renal fibrosis in a subject comprising applying the cell sheet of any one of claims 1 to 10 or 20 to kidney tissue of a subject, thereby suppressing renal fibrosis in the subject. In certain embodiments, applying the cell sheet to the kidney tissue suppresses renal fibrosis to a greater extent than a hBMSC sheet prepared from hBMSCs that are not a clonal cell line. In certain embodiments, the hBMSCs in the cell sheet are allogeneic to the subject. In certain embodiments, the subject is human.

In certain aspects the disclosure relates to a bone marrow-derived mesenchymal stem cell sheet comprising one or more layers of bone marrow-derived mesenchymal stem cells (hBMSCs), wherein the cell sheet is prepared from a human clonal bone marrow-derived mesenchymal stem cell line generated from a single cell, and wherein the cell sheet is treated with interferon gamma (IFN-γ) or basic fibroblast growth factor (bFGF). In some embodiments, the hBMSCs in the cell sheet are confluent. In some embodiments, the cell sheet consists essentially of hBMSCs. In some embodiments, at least 90% of cells in the cell sheet are hBMSCs. In some embodiments, the hBMSCs in the cell sheet exhibit increased expression of one or more proteins selected from the group consisting of HLA-DR, PD-L1, Indoleamine 2,3-dioxygenase (IDO), interleukin 10 (IL-10) and Prostaglandin E2 (PGE-2) relative to an hBMSC sheet that is not treated with IFN-γ or bFGF. In certain aspects the disclosure relates to a composition comprising a cell sheet as described herein and a polymer-coated culture support that is removable from the cell sheet.

In certain aspects the disclosure relates to a method for producing a human bone marrow-derived mesenchymal stem cell (hBMSC) sheet comprising one or more layers of confluent human bone marrow-derived mesenchymal stem cells (hBMSCs), the method comprising:
a) culturing hBMSCs in culture solution on a temperature-responsive polymer which has been coated onto a substrate surface of a cell culture support, wherein the hBMSCs in culture solution are a human clonal bone marrow-derived mesenchymal stem cell line generated from a single cell, and wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened;
c) detaching the cell sheet from the culture support; and
d) culturing the cell sheet in culture solution containing interferon gamma (IFN-γ) or basic fibroblast growth factor (bFGF).

In some embodiments, the culturing step (a) comprises adding hBMSCs to the culture solution at an initial cell seeding density from 4.5×10⁴ to 3.4×10⁵ cells/cm². In some embodiments, the method further comprises culturing the hBMSCs through multiple subcultures prior to the culturing step (a). In some embodiments, 2 to 10 subcultures of the hBMSCs are performed prior to the culturing step (a). In some embodiments, the hBMSCs are cultured in the culture solution on the temperature-responsive polymer for at least 1 day before the adjusting step (b). In some embodiments, the hBMSCs are cultured in the culture solution on the temperature-responsive polymer for 1 to 3 days. In some embodiments, the adjusting step (b) is performed when the hBMSCs in culture solution are confluent. In certain aspects the disclosure relates to a cell sheet produced by a method described herein.

In certain aspects the disclosure relates to a method of transplanting a cell sheet to a subject comprising applying a cell sheet as described herein to a tissue of a subject. In certain aspects the disclosure relates to a method of modulating immune response in a subject comprising applying a cell sheet as described herein to a tissue of a subject. In some embodiments, applying the cell sheet to the tissue results in increased levels in the tissue of one or more proteins selected from the group consisting of HLA-DR, PD-L1, Indoleamine 2,3-dioxygenase (IDO), interleukin 10 (IL-10) and Prostaglandin E2 (PGE-2), relative to a tissue that is not contacted with the cell sheet. In some embodiments, the hBMSCs in the cell sheet are allogeneic to the subject. In some embodiments, the subject is human.

### BRIEF DESCRIPTION OF THE DRAWINGS

FIG. 1 shows that clonal BMSCs from multiple cell lines used for cell sheet fabrication exhibit positive (≥95%) surface antigen expression of phenotypic MSC markers (CD44, CD73, CD90, CD105) and negative expression of resident bone marrow/blood cells (CD31, CD34, CD45). Percentage positive was measured as above 0.5% of fluorescent isotype control. N≥2.

FIG. 2 shows clonal BMSC sheet preparation and their cytokine production. Clonal BMSC sheets engineered from multiple cell lines were successfully prepared on TRCD. (Scale bar; 1 cm). Clonal BMSC sheets exhibited gene expression of multiple tissue regenerative cytokines, such as HGF, VEGF, and FGF2, as well as a heterogeneous, whole BMSC sheet. These data were normalized with the gene expression levels in whole BMSC sheets.

FIG. 3 shows comparison of the gene expression levels of immunomodulatory cytokines as single cells and cell sheets. Comparison of the gene expression levels of IL-10, IDO, and PGE-2 as single cells and cell sheets was investigated by qPCR. Cell sheet formation of clonal BMSCs significantly enhanced gene expression of immunomodulatory cytokines compared to single cell condition. These data were normalized with the expression level of single cells.

FIG. 4 shows human clonal BMSC sheet transplantation in a rat IRI model. This is the schema of an animal study. Five human clonal BMSC sheets are transplanted into the left kidney of immunodeficient rats, and the left renal artery and vein are ischemic for 60 minutes followed by re-perfusion.

FIG. 5 shows engraftment and migration of transplanted human clonal BMSC sheets. These are images of human fibronectin (hFN) staining in rat kidney at 3-days after IRI. The left is IRI without cell sheet transplantation, the middle is IRI with clonal BMSC sheet transplantation onto the renal capsule, and the right is IRI with clonal BMSC sheet transplantation onto the kidney without capsule. In the middle and right images, many hFN-positive cells derived from the clonal BMSC sheets are detected on the top of parenchyma, and some of the cells migrate into renal parenchyma (arrow).

FIG. 6 shows therapeutic effects of human clonal BMSC sheets in a rat IRI model without renal capsule. The upper row shows representative PAS staining images and tubular injury scores of native, IRI, and without capsule clonal BMSC sheets transplantation groups at 3-days after IRI. Tubular injury is indicated by black arrows in the IRI only and clonal MSC sheet groups. The clonal BMSC sheet suppresses the early phase of IRI injury as indicated by the clonal BMSC sheet group's lower tubular injury scores compared to the IRI group. The lower row shows representative MT staining images and the graph of collagen positive blue area fraction, indicating fibrotic areas, of Native, IRI, WB, without capsule clonal BMSC sheet transplantation group at 28-days after IRI. The blue area of MT staining indicates collagen deposition (yellow arrow). Clonal BMSC sheet transplantation without capsule shows the highest inhibition ability of fibrosis compared to IRI and WB sheet group, as indicated by lowest collagen positive blue area.

FIG. 7 shows therapeutic effects of human clonal BMSC sheets in a rat IRI model with renal capsule. The upper row shows representative PAS staining images and tubular injury scores of native, IRI, and with capsule clonal BMSC sheets transplantation groups at 3-days after IRI. Tubular injury is indicated by black arrows in the IRI only and clonal MSC sheet groups. The clonal BMSC sheet suppresses the early phase of IRI injury as indicated by the clonal BMSC sheet group's lower tubular injury scores compared to the IRI group. The lower row shows representative MT staining images and the graph of collagen positive blue area fraction, indicating fibrotic areas, of Native, IRI, WB, with capsule clonal BMSC sheet transplantation group at 28-days after RI. The area of MT staining indicates collagen deposition (yellow arrow). Clonal BMSC sheet transplantation with capsule shows the highest inhibition ability of fibrosis compared to IRI and WB sheet group, as indicated by lowest collagen positive blue area.

FIG. 8 shows clonal BMSC sheet fabrication with different conditions (density and culture time). Clonal BMSC sheets were detached as a sheet form from the initial cell density of 0.8 million cells/35 mm diameter TRCD at 6 hours, 0.6 million cells/35 mm TRCD at 1 day, and 0.4 million cells/35 mm diameter TRCD at 3 and 6 days after seeding.

FIG. 9 shows gene expression levels of clonal BMSC suspension and sheets related to cytokine secretion ability. Gene expression levels (HGF, IL10, VEGF) of clonal BMSC sheets were compared with gene expression levels of single clonal BMSC suspension (SC). Clonal BMSC sheets showed the higher gene expression levels related to HGF, IL10, VEGF cytokine secretion, compared to single cell suspension of clonal BMSCs (SC). Furthermore clonal BMSC sheets prepared with the density of 1.3 million cells/35 mm TRCD expressed higher gene expression levels (HGF, IL10, VEGF), compared to gene expression levels of clonal BMSC sheets prepared with the initial cell density of 0.4 million cells/35 mm TRCD.

FIG. 10 shows cytokine amounts secreted from clonal BMSC sheets. Cytokine amounts secreted from clonal BMSC sheets with different initial cell density (0.6, 1.5, 3 million cells/35 mm TRCD) were detected. Higher initial cell density groups (1.5 and 3 million cells/35 mm TRCD) secreted higher amount of cytokines per cell sheet and cell, compared to 0.6 million cells/35 mm TRCD group.

FIG. 11 shows high initial cell adhesion ability in frozen cells. Adherent fresh and frozen cell incubation were observed after 15- or 30-minute incubation. Frozen cells showed high initial and mature cell adhesion ability compared to fresh cells. (Scale bar; 200 μm).

FIG. 12 shows cell sheet preparation using fresh and frozen cells. Cell sheets engineered from fresh and frozen cells in multiple initial seeding densities were shown. Cell sheets containing larger numbers of the cells showed lager cell sheet shapes. Frozen cells enabled preparation of cell sheets at lower initial cell density compared to fresh cells. (Scale bar; 1 cm).

FIG. 13 shows cytokine production in cell sheets prepared from fresh or frozen cells at various initial cell densities. Cytokine production of each cell sheet is shown. Cell sheets prepared from frozen cells at each initial cell density showed cytokine production comparable to cell sheets prepared from fresh cells, suggesting that frozen cells can be an option for cell sheet production. In particular, frozen cells are beneficial to perform quick cell sheet production based on their high initial cell adhesion ability.

FIG. 14 shows that clonal BMSC sheets upregulate expression of immunomodulatory molecules in response to interferon gamma (IFN-γ). Clonal BMSC sheets exhibited peak upregulation of immunomodulatory genes when exposed to 25 ng/mL IFN-γ. Gene expressions were represented as fold change normalized to a non-primed control cell sheet under the same conditions. (Sample number; n=2).

FIG. 15 shows that cell sheets fabricated with IFN-γ supplementation exhibit upregulation of immunomodulatory molecules. Prior to cell sheet detachment, 25 ng/mL IFN-γ was added at 2 days. Clonal BMSC sheets primed for 2-days prior to detachment exhibited significant increase in gene expressions of HLA-DR, PD-L1, and IDO. Gene expressions were represented as fold change normalized to respective non-primed control cell sheet fabricated under the same conditions. (Sample number; n=6).

FIG. 16 shows the influence of IFN-γ priming duration on clonal BMSC sheet immunomodulatory gene expression. Increased duration of IFN-γ priming was associated with further increased gene expression of immunomodulatory molecules. Clonal BMSC sheets fabricated after 4-days and 6-days of IFN-γ priming exhibited upregulation of immunomodulatory genes (HLA-DR, PD-L1, IDO, IL-10, and PGE-2) with highest expression after 6-days of priming. Gene expression was represented as fold change normalized to respective non-primed control cell sheet fabricated under the same conditions. (Sample number; n=6).

FIG. 17 shows the stability of IFN-γ priming effect on clonal BMSC sheet gene expression. Cell sheets fabricated with either 6-days, 4-days, 2-days, or 0-days IFN-γ supplementation were replated in normal culture conditions for 4-days to analyze stability of IFN-γ effect. The results indicated that even after removal of IFN-γ clonal BMSCs continue to exhibit upregulated gene expression of HLA-DR, PD-L1, IDO, and IL-10. Clonal cell sheets fabricated with 4-days of IFN-γ priming exhibited highest gene expression of immunosuppressive (IDO, PD-L1, and IL-10) molecules. Gene expression was represented as fold change normalized to respective non-primed control cell sheet fabricated under the same conditions. (Sample number; n=4).

FIG. 18 shows cell sheets fabricated with basic fibroblast growth factor (bFGF) supplementation exhibit upregulation of immunomodulatory molecules. Prior to cell sheet detachment, 5 or 80 ng/mL bFGF was added at 2 days. Clonal BMSC sheets primed for 2-days prior to detachment exhibited a significant increase in HLA-DR, IDO, and IL-10. Gene expression was represented as fold change normalized to respective non-primed control cell sheet fabricated under the same conditions. (Sample number; n=3).

### DETAILED DESCRIPTION OF THE INVENTION

This disclosure describes preparation and properties for human bone marrow-derived mesenchymal stem cell (hBMSC) sheets for treating renal disorders. Currently, injected MSC cell suspensions are harvested using enzymes that compromise MSC functions and engraftment capabilities, resulting in low tissue retention and survival, and sub-optimal therapeutic properties. Cell sheets created without enzymes and as living sheets with an extracellular matrix (ECM) and cell receptors intact, such as those described herein, can be physically placed on tissue sites with highly improved retention and engraftment efficiencies.

Human clonal bone marrow-derived mesenchymal stem cells (hBMSCs) derived from a single cell were used to prepare cell sheets in vitro in temperature-responsive cell culture dishes (TRCDs) coated with a temperature-responsive polymer. Confluent cell sheets formed at 1-3 days after seeding and were detached from the TRCD by cooling the cultures to room temperature. The hBMSC sheets produced by these methods suppressed renal fibrosis in a rat ischemia-reperfusion injury model to a greater extent than hBMSC sheets prepared from hBMSCs that are not a clonal cell line.

I. Human Bone Marrow-Derived Mesenchymal Stem Cells (hBMSCs)

The term “human bone marrow-derived mesenchymal stem cell” or “hBMSC” as used herein refers to a mesenchymal stem cell that has been isolated from human bone marrow.

The term “clonal cell line” as used herein refers to a cell line that is generated from a colony grown from a single cell. For example, the term “human clonal bone marrow-derived mesenchymal stem cell line” or “human clonal BMSC line” as used herein refers to a hBMSC cell line that is generated from a colony grown from a single hBMSC. Non-clonal cultured BMSCs, even when derived from a single donor, are intrinsically heterogeneous populations composed of highly diverse BMSC subsets with variable surface marker expression, colony formation, differentiation potency, immunomodulatory/regenerative potentials and in vivo behavior. This disclosure describes human clonal BMSC lines generated from a single cell-derived colony to overcome this limitation on heterogeneity and enhance the therapeutic effects of BMSC-based therapy through rational clonal selection, eliminating the variability between donors and the cell product produced.

Methods for preparing human clonal BMSC lines are known in the art and are described, for example, in U.S. Pat. No. 7,781,211, which is incorporated by reference herein in its entirety. In some embodiments, human clonal BMSC lines may be prepared from a biological sample of bone marrow by (i) allowing the biological sample to settle by gravity in a first container producing a first supernatant of lower density cells; (ii) transferring the first supernatant directly without undergoing centrifugation to a second container of growth medium and allowing cells to settle to the bottom producing a second supernatant of lower density cells; (iii) transferring the second supernatant directly without undergoing centrifugation to a third container of growth medium and allowing cells to settle to the bottom, producing a third supernatant of lower density cells; (iv) transferring the third supernatant directly without undergoing centrifugation to another container of growth medium and allowing cells to settle to the bottom, producing another supernatant of lower density cells; (v) allowing single-cell derived colonies to appear on the bottom of the container of step (iv); (vi) isolating the single-cell derived colonies; and (vii) expanding cells from the single-cell derived colonies in a further other container of growth medium to obtain a homogeneous population of single cell-derived clonal bone marrow cells.

The hBMSC sheets described herein differ from harvested hBMSC suspensions in several ways. Suspensions of hBMSCs contain single cells that do not have an ECM or cell-cell junctions because the adhesive proteins in these cell-cell junctions must be disrupted (e.g. by trypsin treatment) to harvest cells from culture surfaces for preparation of the cell suspension culture. In contrast to single cell suspensions of hBMSCs, the hBMSC sheets described herein contain both an ECM and cell-cell junctions among the hBMSCs that are generated during formation of the cell sheet. The intact ECM and cell-cell junctions facilitate adhesion of the hBMSC sheet to target tissue during transplantation to a host organism.

II. Cell Sheets Produced from hBMSCs

In certain aspects the present disclosure relates to a human bone marrow-derived mesenchymal stem cell sheet comprising one or more layers of confluent human bone marrow-derived mesenchymal stem cells (hBMSCs), wherein the cell sheet is prepared from a human clonal bone marrow-derived mesenchymal stem cell line generated from a single cell. The term “human bone marrow-derived mesenchymal stem cell sheet” or “hBMSC sheet” as used herein refers to a cell sheet obtained by growing human bone marrow-derived mesenchymal stem cell on a cell culture support in vitro. The hBMSC sheets described herein are harvested as a sheet of one or more layers with a temperature shift using a temperature-responsive culture dish (TRCD) without any enzyme treatment. The hBMSC sheets maintain their size and shape by retaining tissue-like structures, actin filaments, extracellular matrix, intercellular proteins, and high cell viability, all of which are related to improved cell survival and cellular functions relevant to cell therapy. Accordingly, the cell sheets described herein may comprise structural features that improve cell survival and cell function, including an extracellular matrix, cell adhesion proteins and cell junction proteins. Thus, the hBMSC sheets prepared by the methods described herein have several beneficial characteristics compared to MSCs produced by other methods. For example, chemical disruption (proteolytic enzyme treatment) is widely used in cells harvested for stem cell therapy. However, the chemical disruption method is unable to maintain tissue-like structures of cells as well as cell-cell communication, since enzyme treatment disrupts the extracellular and intracellular proteins (cell-cell and cell-ECM junctions). Accordingly, protein cleavage by enzymes reduces cell viability and cellular functions relevant to cell therapy.

In some embodiments, the cell sheet consists of hBMSCs. In some embodiments, the cell sheet consists essentially of hBMSC. In some embodiments, at least 50%, 60%, 70%, 80%, 90%, 95%, 96%, 97%, 98% or 99% of cells in the cell sheet are hBMSC. In some embodiments, 100% of the cells in the cell sheet are hBMSC.

The hBMSC may be added to the culture solution on the temperature-responsive polymer in the cell culture support at various cell densities to optimize formation of the cell sheet or its characteristics. For example, cytokine expression levels in the hBMSC may be optimized by controlling the initial cell density of the hBMSC in the cell culture support (e.g. TRCD). In some embodiments, increasing the initial cell density of the hBMSCs in the cell culture support increases cytokine expression (e.g., one or more of hepatocyte growth factor (HGF), vascular endothelial growth factor (VEGF) and interleukin 10 (IL-10)). In some embodiments, decreasing the initial cell density of the hBMSCs in the cell culture support decreases cytokine expression. In some embodiments the initial cell density of the hBMSCs in the cell culture support used for preparation of the cell sheet is from 0.5×10⁴/cm² to 9×10⁵/cm². In some embodiments, the initial cell density of the hBMSCs in the cell culture support is at least 0.5×10⁴, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, or 9×10⁵ cells/cm². Any of these values may be used to define a range for the initial cell density of the hBMSCs in the cell culture support. For example, in some embodiments, the initial cell density in the cell culture support is from 4.5×10⁴ to 3.4×10⁵ cells/cm² (4×10⁵ to 3×10⁶ cells/35 mm diameter UpCell dish which has an area of 8.8 cm²).

The hBMSC sheets described herein may be transplanted to a target tissue in a host organism (e.g. a human) for therapeutic uses. Transplantation of the hBMSC sheets to the target tissue may result in the formation of capillaries (angiogenesis) in the host tissue, as well as blood vessel formation between the transplanted cell sheet and the host tissue. This neocapillary formation is an important capability for sheet engraftment, cell viability and tissue regeneration. In addition, this new blood vessel recruitment into sheets on the target tissue suggests that implanted hBMSC sheets continually secrete paracrine factors to modulate engraftment.

In some embodiments, the hBMSC sheets express one or more cytokines, for example, one or more immunomodulatory factors (e.g., Interleukin-10 (IL-10);), anti-fibrotic factors (e.g., hepatocyte growth factor (HGF); Bone morphogenetic protein 7 (BMP-7)), and/or angiogenic factors (e.g., Vascular endothelial growth factor (VEGF); basic fibroblast growth factor (bFGF)). In some embodiments the cytokine is selected from hepatocyte growth factor (HGF), fibroblast growth factor (FGF), vascular endothelial growth factor (VEGF), interleukin-10 (IL-10), prostaglandin E2 (PGE-2), bone morphogenetic protein 7 (BMP-7), and basic fibroblast growth factor (bFGF). In some embodiments, expression of the cytokine (e.g., an immunomodulatory factor, anti-fibrotic factor, or angiogenic factor) in the cell sheet is increased relative to a suspension of hBMSCs containing an equivalent number of cells, or relative to a cell sheet prepared from hBMSCs that are not a clonal cell line. In some embodiments, expression of the cytokine (e.g., an immunomodulatory factor, anti-fibrotic factor, or angiogenic factor) is decreased relative to a suspension of hBMSCs containing an equivalent number of cells, or relative to a cell sheet prepared from hBMSCs that are not a clonal cell line.

The hBMSC sheets described herein may continue to express cytokines after transplantation to a target tissue in a host organism. In some embodiments, the cell sheet expresses the cytokine for at least 1, 2, 3, 4, 5, 6, 7, 8, 9, 10, 15, 20, 25 or 30 days after transplantation to a tissue (e.g., kidney tissue) in a host organism. In some embodiments, the cell sheet expresses the cytokine for at least 1, 2, 3, 4, 5 or 6 months after transplantation to a tissue (e.g., kidney tissue) in a host organism.

Current stem cell therapies often use cultured stem cells isolated from biopsies as injectable cell suspensions (Bayoussef et al., 2012, J Tissue Eng Regen Med, 6(10)). Injected cell suspensions typically exhibit lower engraftment into and retention within diseased organs or tissues (Devine et al., 2003, Blood, 101(8), 2999-3001). Loss of intact ECM and cell-cell junctions (i.e., communication) in stem cell suspensions through enzymatic disruption at harvest compromises stem cell function, engraftment and survival in vivo, and can limit therapeutic efficacy in vivo. In contrast, the methods of preparing hBMSC sheets described herein preserve intrinsic cell functional structures, improving attachment of the cell sheet to the target tissue after transplantation. For example, in some embodiments, the cell sheet remains attached to the target tissue (e.g., kidney tissue) in the host organism for at least 1, 2, 3, 4, 5, 6, 7, 8, 9, 10, 15, 20, 25 or 30 days after transplantation to a tissue in a host organism. In some embodiments, the cell sheet remains attached to the target tissue (e.g., kidney tissue) in the host organism for at least 1, 2, 3, 4, 5 or 6 months after transplantation to a tissue of a host organism.

III. Methods for Producing hBMSC Sheets In Vitro

In certain aspects, the present disclosure relates to a method for producing a human bone marrow-derived mesenchymal stem cell sheet comprising one or more layers of confluent human bone marrow-derived mesenchymal stem cells (hBMSCs), the method comprising:
a) culturing hBMSCs in culture solution on a temperature-responsive polymer which has been coated onto a substrate surface of a cell culture support, wherein the hBMSCs in culture solution are a human clonal bone marrow-derived mesenchymal stem cell line generated from a single cell, and wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

General methods for preparing cell sheets are known in the art and are described, for example, in U.S. Pat. Nos. 8,642,338; 8,889,417; 9,981,064; and 9,114,192, each of which is incorporated by reference herein in its entirety.

The temperature-responsive polymer used to coat the substrate of the cell culture support has an upper or lower critical solution temperature in aqueous solution which is generally in the range of 0° C. to 80° C., for example, 10° C. to 50° C., 0° C. to 50° C., or 20° C. to 45° C.

The temperature-responsive polymer may be a homopolymer or a copolymer. Exemplary polymers are described, for example, in Japanese Patent Laid-Open No. 211865/1990. Specifically, they may be obtained by homo- or co-polymerization of monomers such as, for example, (meth)acrylamide compounds ((meth)acrylamide refers to both acrylamide and methacrylamide), N-(or N,N-di)alkyl-substituted (meth)acrylamide derivatives, and vinyl ether derivatives. In the case of copolymers, any two or more monomers, such as the monomers described above, may be employed. Further, those monomers may be copolymerized with other monomers, one polymer may be grafted to another, two polymers may be copolymerized, or a mixture of polymer and copolymer may be employed. If desired, polymers may be crosslinked to an extent that will not impair their inherent properties.

The substrate which is coated with the polymer may be of any types including those which are commonly used in cell culture, such as glass, modified glass, polystyrene, poly(methyl methacrylate), polyesters, and ceramics.

Methods of coating the support with the temperature-responsive polymer are known in the art and are described, for example, in Japanese Patent Laid-Open No. 211865/1990. Specifically, such coating can be achieved by subjecting the substrate and the above-mentioned monomer or polymer to, for example, electron beam (EB) exposure, irradiation with 7-rays, irradiation with UV rays, plasma treatment, corona treatment, or organic polymerization reaction. Other techniques such as physical adsorption as achieved by coating application and kneading may also be used.

The coverage of the temperature responsive polymer may be in the range of 0.4-3.0 μg/cm², for example, 0.7-2.8 μg/cm², or 0.9-2.5 μg/cm². The morphology of the cell culture support may be, for example, a dish, a multi-plate, a flask or a cell insert.

The cultured cells may be detached and recovered from the cell culture support by adjusting the temperature of the support material to the temperature at which the polymer on the support substrate hydrates, whereupon the cells can be detached. Smooth detachment can be realized by applying a water stream to the gap between the cell sheet and the support. Detachment of the cell sheet may be affected within the culture solution in which the cells have been cultivated or in other isotonic fluids, whichever is suitable. In some embodiments, the hBMSCs are cultured in the culture solution on the temperature-responsive polymer for at least 12 hours, at least 24 hours, at least 1 day, at least 2 days, at least 3 days, at least 4 days, at least 5 days, at least 6 days, or at least 7 days before adjusting the temperature of the culture solution to below the lower critical solution temperature for release of the cell sheet from the support material. In some embodiments, the hBMSCs are cultured in the culture solution on the temperature-responsive polymer for fewer than 2 days, fewer than 3 days, fewer than 4 days, fewer than 5 days, fewer than 6 days, fewer than 7 days before adjusting the temperature of the culture solution to below the lower critical solution temperature for release of the cell sheet from the support material. In some embodiments, the hBMSCs are cultured in the culture solution on the temperature-responsive polymer for about 12 hours, about 24 hours, about 1 day, about 2 days, about 3 days, about 4 days, about 5 days, about 6 days, or about 7 days before adjusting the temperature of the culture solution to below the lower critical solution temperature for release of the cell sheet from the support material. Any of these values may be used to define a range for the length of time in with the hBMSCs are cultured in the culture solution. For example, in some embodiments, the hBMSCs are cultured in the culture solution for 1 to 2 days, 1 to 3 days, or 1 to 4 days.

In a particular embodiment, the temperature-responsive polymer is poly(N-isopropyl acrylamide) Poly(N-isopropyl acrylamide) has a lower critical solution temperature in water of 31° C. If it is in a free state, it undergoes dehydration in water at temperatures above 31° C. and the polymer chains aggregate to cause turbidity. Conversely, at temperatures of 31° C. and below, the polymer chains hydrate to become dissolved in water, thereby causing release of the cell sheet from the polymer. In a particular embodiment, this polymer covers the surface of a substrate such as a Petri dish and is immobilized on it, for example, by chemical or physical grafting or tethering. Therefore, at temperatures above 31° C., the polymer on the substrate surface also dehydrates but since the polymer chains cover the substrate surface and are immobilized on it, the substrate surface becomes hydrophobic with polymer dehydration. Conversely, at temperatures of 31° C. and below, the polymer on the substrate surface hydrates but since the polymer chains cover the substrate surface and are immobilized on it, the substrate surface becomes hydrophilic with polymer dehydration. The hydrophobic surface is an appropriate surface for the adhesion and growth of cells, whereas the hydrophilic surface inhibits the adhesion of cells and the cells are detached simply by cooling the culture solution.

Culture solutions for mesenchymal stem cells are known in the art and are described, for example, in U.S. Pat. Nos. 9,803,176 and 9,782,439, each of which is incorporated by reference herein in its entirety. In some embodiments, the culture solution comprises human platelet lysate (hPL). In some embodiments, the culture solution comprises ascorbic acid. In some embodiments, the culture solution contains at least one product obtained from a non-human animal (e.g. FBS). In some embodiments, the culture solution does not contain a product obtained from a human. In a particular embodiment, the culture solution comprises one or more of Dulbecco's Modified Eagle's Medium (DMEM) (Life Technologies, CA, USA), Fetal Bovine Serum (FBS) (Thermo Fisher Scientific), MycoZap Prophylactic (Lonza), and an antibiotic, e.g., penicillin streptomycin.

The hBMSC sheet may be prepared in a range of different sizes depending on the application. In some embodiments, the hBMSC sheet has a diameter of at least 1, 2, 3, 4, 5, 6, 7, 8, 9, 10, 15 or 20 cm. Any of these values may be used to define a range for the size of the hBMSCs sheet. For example, in some embodiments, the hBMSC sheet has a diameter from 1 to 20 cm, from 1 to 10 cm or from 2 to 10 cm. In some embodiments, the hBMSC sheet has an area of at least 1, 2, 3, 4, 5, 6, 7, 8, 9, 10, 15, 20, 30, 40, 50, 60, 70, 80, 90, 100, 150, 200, 250 or 300 cm². Any of these values may be used to define a range for the size of the hBMSC sheet. For example, in some embodiments, the hBMSC sheet has an area from 1 to 100 cm², 3 to 70 cm², or 1 to 300 cm². The methods described herein result in an hBMSC sheet in which the surface area and/or diameter of the hBMSC sheet is much greater than its thickness. For example, in some embodiments the ratio of the surface area of the hBMSC sheet to its thickness is at least 10:1, 100:1, 1000:1, or 10,000:1. In some embodiments the ratio of the diameter of the hBMSC sheet to its thickness is at least 10:1, 100:1, 1000:1, or 10,000:1. The hBMSC sheets described herein comprise one or more layers of confluent human bone marrow-derived mesenchymal stem cells (hBMSCs), for example, 1, 2, 3, 4, 5, 6, 7, 8, 9 or 10 layers of hBMSCs. In some embodiments, the hBMSC sheet comprises fewer than 1, 2, 3, 4, or 5 layers of hBMSCs. In some embodiments, the hBMSC sheet comprises at least 1, 2, 3, 4, or 5 layers of hBMSCs.

IV. Methods for Transplanting Human Bone Marrow-Derived MSC (hBMSC) Sheets to a Subject

The hBMSC sheets described herein can be transplanted to a subject by applying the cell sheet to a tissue (e.g., kidney tissue) in the subject. For example, as disclosed in Example 2 below, transplantation of a hBMSC sheet prepared by the methods described herein highly suppressed renal fibrosis in a rat ischemia-reperfusion injury model compared to a hBMSC sheet that was prepared from hBMSCs that are not a clonal cell line.

Accordingly, in some aspects, the present disclosure relates to a method of transplanting a cell sheet to a subject comprising applying a cell sheet as described herein to a tissue of a subject. In some embodiments, the tissue is kidney tissue. In a particular embodiment, the subject is a human. One of the advantages of the hBMSC sheets described herein is that the extracellular matrix of the cell sheet act as an adhesive to bind the cell sheet to the tissue of the subject, such that stitching is not required to adhere the cell sheet to the tissue. A support membrane may be used to transfer the harvested hBMSC sheet released from the culture surface to the tissue of the subject. The support membrane for such transfer can be, for example, poly(vinylidene difluoride) (PVDF), cellulose acetate, and cellulose esters. The hBMSC sheets readily adhere to target tissue, self-stabilizing without suturing after being placed directly onto the target tissue for a short period of time. For example, in some embodiments, the hBMSC sheet adheres to the target tissue within 5, 10, 15, 20, 25, or 30 minutes after contact with the tissue. In certain embodiments, the hBMSC in the cell sheet are allogeneic to the subject, i.e. are isolated from a different individual from the same species as the subject, such that the genes at one or more loci are not identical. In certain reported cases, MSCs seemingly avoid allogeneic rejection in humans and in animal models (Jiang et al., 2005, Blood, 105(10), 4120-4126). Thus the hBMSC sheets described herein may be used in allogeneic cell therapies as an off-the-shelf product, avoiding the unfavorable costs and development disincentives associated with autologous stem cell treatment methods.

In some embodiments, the hBMSC cell sheet is transplanted to a subject that has received a kidney transplant. In some embodiments, the hBMSC cell sheet is a transplanted to a subject that has an acute kidney injury. In some embodiments, the hBMSC cell sheet is transplanted to a subject that has an ischemia re-perfusion injury. In some embodiments, the hBMSC cell sheet is a transplanted to a subject that has a kidney fibrosis.

In certain aspects, the disclosure relates to a method of suppressing renal fibrosis (e.g. fibrosis of the renal cortex) in a subject comprising applying a hBMSC cell sheet as described herein to kidney tissue of a subject, thereby suppressing renal fibrosis (e.g. fibrosis of the renal cortex) in the subject.

In certain aspects, the disclosure relates to a method of treating kidney tubule injury in a subject comprising applying a hBMSC cell sheet as described herein to kidney tissue of a subject, thereby treating kidney tubule injury in the subject.

### EXAMPLES

### Example 1. Evaluation of Human Clonal BMSCs and Preparation of Human Clonal BMSC Sheets

A human clonal BMSC library was established from donor bone marrow by SCM Lifescience (South Korea) using a subfractionation culture method. See Song, S. U. et al. 2008, Stem Cells Dev 17, 451-461, doi:10.1089. Isolated human clonal BMSCs were cultured in Dulbecco's Modified Eagle's Medium (DMEM) (Life Technologies, CA, USA) supplemented with 10% Fetal Bovine Serum (FBS) (Thermo Fisher Scientific), 0.1% MycoZap Prophylactic (Lonza), and 1% penicillin streptomycin at 37° C., 5% CO₂ incubator. Banked clonal BMSCs were verified by testing for: CD73+, CD90+, CD105+, CD44+, CD34−, CD31−, CD45−. Each clonal BMSC line was revived from the established passage 8 bank and culture through passage 10 at 1,000-2,000 cells/cm². Cells were collected via enzymatic detachment and single-stained for flow cytometry (BD Canto) at the University of Utah Flow Core.

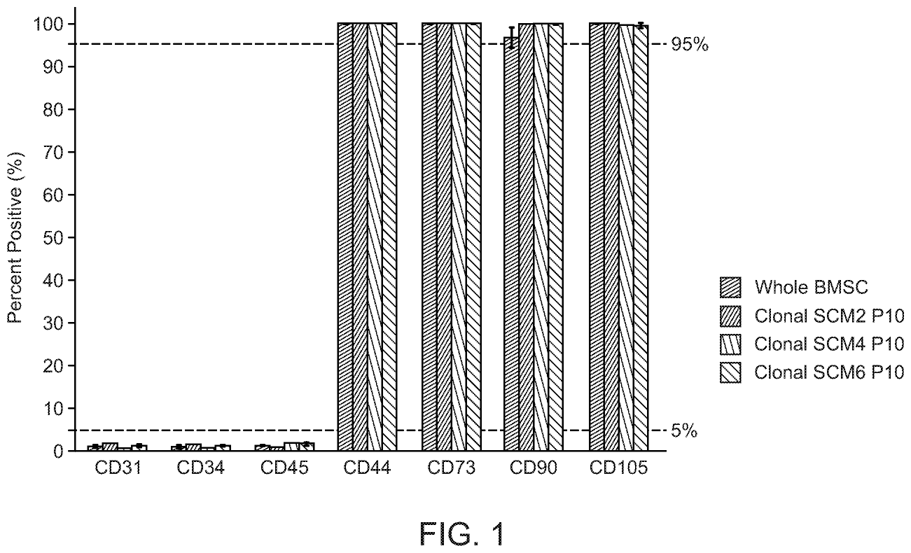

As shown in FIG. 1, clonal BMSCs from multiple cell lines used for cell sheet fabrication exhibited positive (≥95%) surface antigen expression of phenotypic MSC markers (CD44, CD73, CD90, CD105) and negative expression of resident bone marrow/blood cells (CD31, CD34, CD45). Percentage positive was measured as above 0.5% of fluorescent isotype control. N≥2.

Banked clonal BMSCs were verified by testing for: trilineage potential (osteogenic, adipogenic, chondrogenic), and for surface antigen expression (CD73+, CD90+, CD105+, CD44+, CD34−, CD31−, CD45−).

Banked clonal BMSCs were subcultured and seeded on 35-mm temperature responsive culture dishes (TRCD) and cultured for 6-days at passage 10. Clonal BMSC sheets using different clonal BMSC cell lines were harvested from the cell culture surface at room temperature and employed for qPCR to investigate their cytokine production at the level of gene expression.

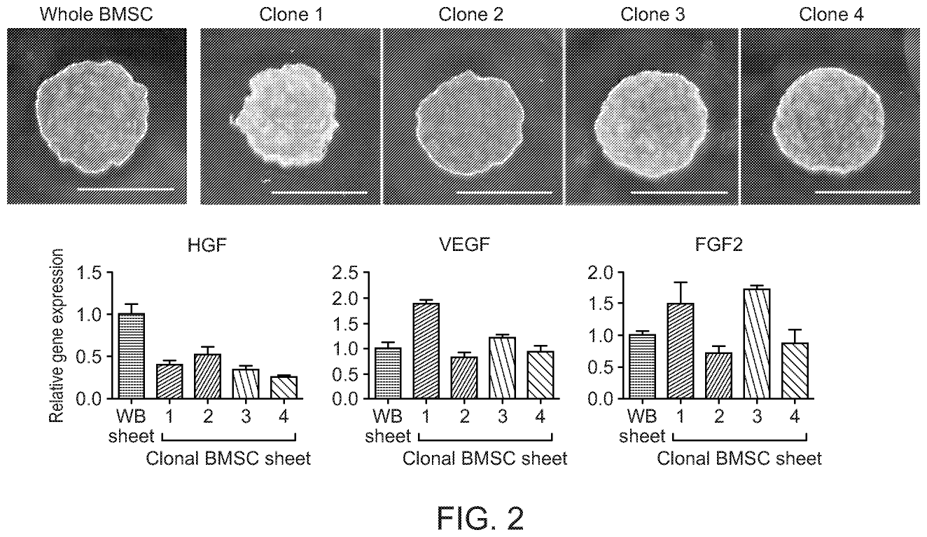

As shown in FIG. 2, the clonal BMSC sheets engineered from multiple cell lines were successfully prepared on TRCD. Clonal BMSC sheets exhibited gene expression of multiple tissue regenerative cytokines, such as HGF, VEGF, and FGF2. Cell sheets prepared from heterogeneous non-clonal BMSCs (“whole BMSC” in FIG. 2) also produced similar levels of HGF, VEGF, and FGF2.

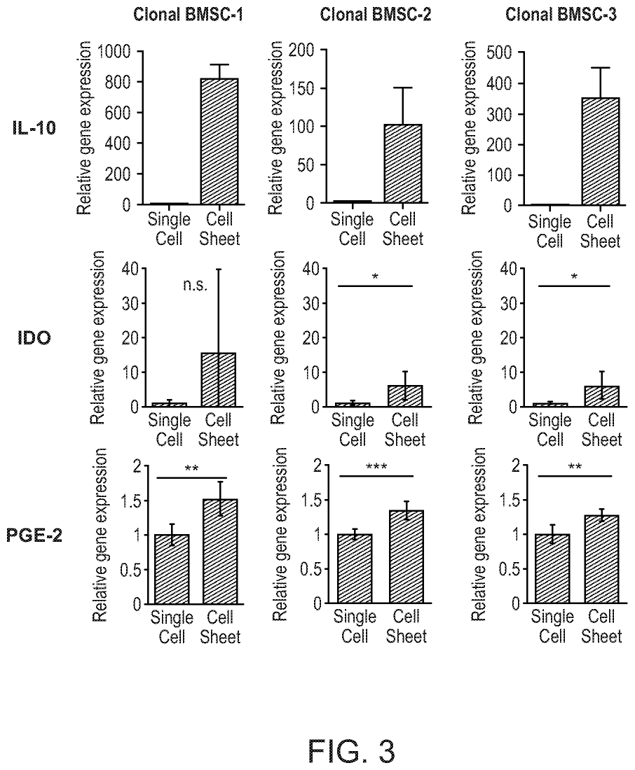

Banked clonal BMSCs were subcultured and harvested from a cell culture dish as single cells at passage 10. Clonal BMSCs were also seeded on 35-mm temperature responsive culture dishes (TRCD) and cultured for 6-days at passage 10 to prepare cell sheets. Multiple cell lines of clonal BMSCs as single cells and cell sheets were prepared for qPCR to investigate their cytokine productions at gene expression levels. Specifically, comparison of the gene expression levels of IL-10, IDO, and PGE-2 as single cells and cell sheets was investigated by qPCR. Cell sheet formation of clonal BMSCs significantly enhanced gene expression of immunomodulatory cytokines compared to single cells. See FIG. 3.

### Example 2. Human Clonal BMSC Sheet Transplantation in a Rat Renal Ischemia-Reperfusion Injury (IRI) Model

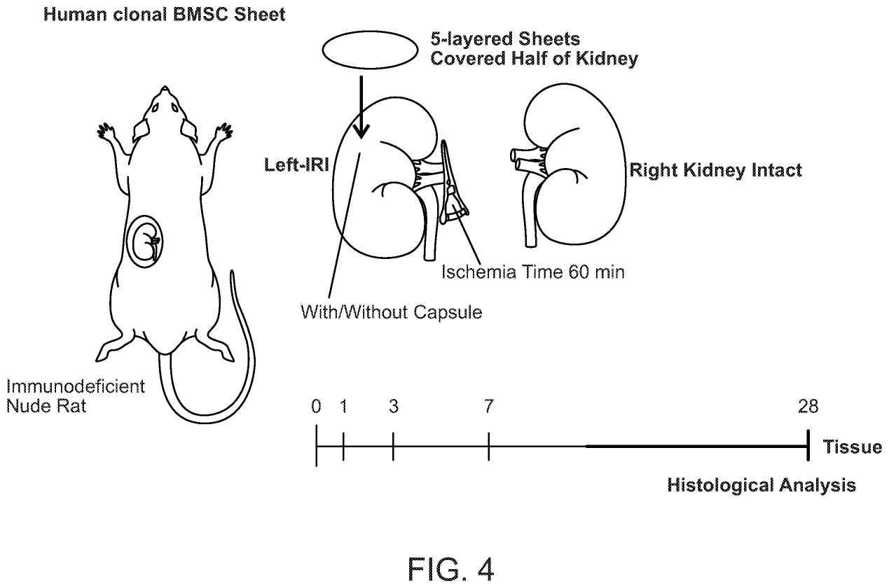

Under isoflurane anesthesia, left kidney of immunocompromised rats were carefully stripped of surrounding fat, and the left renal artery and vein were clamped with a vascular clip for 60 minutes to block their blood flow. After 60 minutes of clamping, the clip was removed, and blood flow was re-perfused to induce renal parenchyma injury. Five human clonal BMSC sheets were transplanted on the renal surface with or without renal capsule. After cell sheet transplantation, IRI kidneys were harvested and analyzed on days 3, and 28. An overview of this study is provided in FIG. 4.

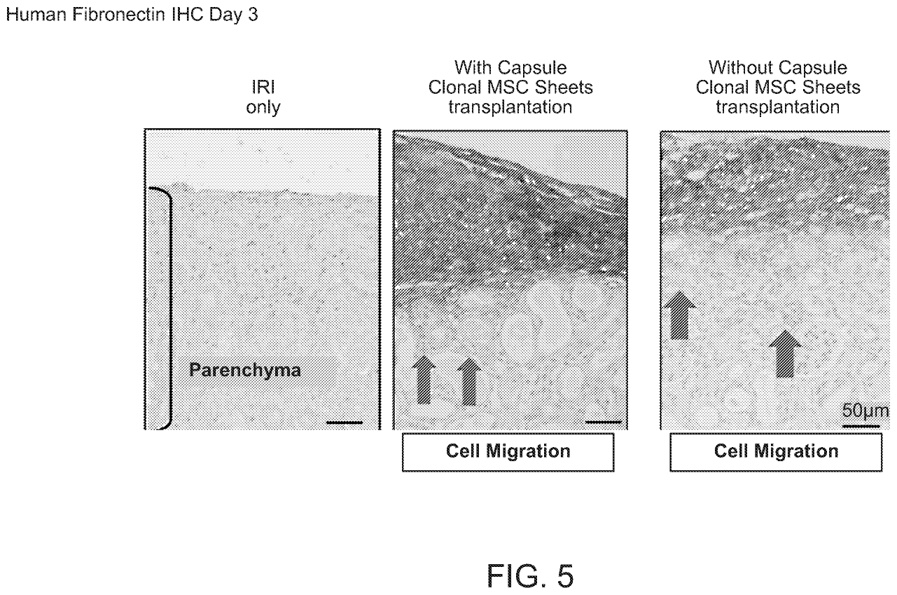

Rat kidneys were harvested on day 3 after IRI, fixed in 4% PFA and embedded in paraffin for histological analysis. Sections were immunolabeled using a specific antibody to human fibronectin and detected using an avidin-biotinylated peroxidase complex. FIG. 5 provides images of human fibronectin (hFN) staining in rat kidney at 3-days after IRI. The left is IRI without cell sheet transplantation, the middle is IRI with clonal BMSC sheet transplantation onto the renal capsule (the thin membranous sheath that covers the outer surface of each kidney), and the right is IRI with clonal BMSC sheet transplantation onto the kidney in which the capsule was removed. In the middle and right images, many hFN-positive cells derived from the clonal BMSC sheets were detected on the top of the parenchyma, and some of the cells migrated into the renal parenchyma (arrow) for both kidneys containing a capsule and for kidneys in which the capsule was removed. These results indicate that cell sheet transplantation was successful with our without the renal capsule. These results also indicate that that even with the renal capsule intact, cells from the transplanted cell sheet were able to pass through capsule and migrate into the parenchyma. This migration of cells form the clonal BMSC cell sheet through the capsule and into the parenchyma would allow for secretion of tissue regenerative cytokines in these tissues and contribute to tissue regeneration.

Rat kidneys harvested at 3- or 28-days after IRI, fixed in 4% PFA and embedded in paraffin were stained with Periodic acid and Schiffs (PAS) and Masson Trichrome (MT) to evaluate renal fibrosis. The degree of tubular injury was assessed based on established scoring methods. See Solez, K., et al., 1970, Medicine 58.5; and Kelleher et al., 1987, Kidney international 31.3. In brief, we graded the extent of tubular necrosis, urinary casts, brush border loss, and tubular dilatation as follows: 0:<10%; 0.5:10-25%; 1:25-45%, 1.5:45-75%, and 2:>75%. The blue area fraction was measured from images of MT staining and analyzed using ImageJ software in each group: Native (healthy normal kidney with capsule, without injury, and without a clonal BMSC cell sheet), IRI only, without capsule non-clonal BMSC (WB) sheet, without capsule clonal BMSC sheets.

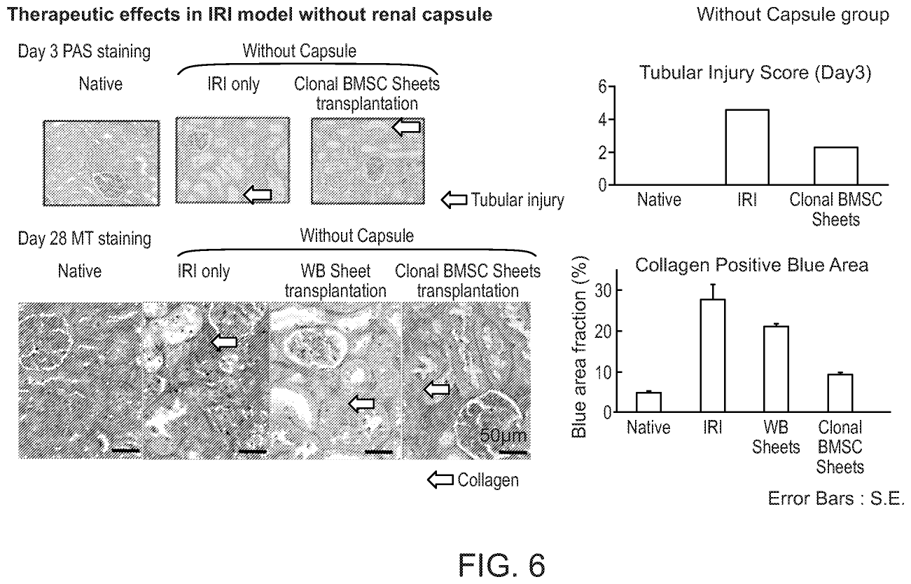

FIG. 6 shows cell sheet transplantation without kidney capsule. In FIG. 6, the upper row shows representative PAS staining images and tubular injury scores of native, IRI, and without capsule clonal BMSC sheets transplantation groups at 3-days after IRI. Tubular injury is indicated by black arrows in the IRI only and clonal MSC sheet groups. The clonal BMSC sheet suppressed the early phase of IRI injury as indicated by the clonal BMSC sheet group's lower tubular injury scores compared to the IRI group. The lower row shows representative MT staining images and the graph of collagen positive fraction, indicating fibrotic areas, of Native, IRI, non-clonal BMSC sheet (WB), without capsule clonal BMSC sheet transplantation group at 28-days after IRI. The area of MT staining indicates collagen deposition (arrow). Clonal BMSC sheet transplantation without capsule showed the highest inhibition of fibrosis compared to the IRI and non-clonal BMSC sheet (WB) group, as indicated by the lowest collagen staining area.

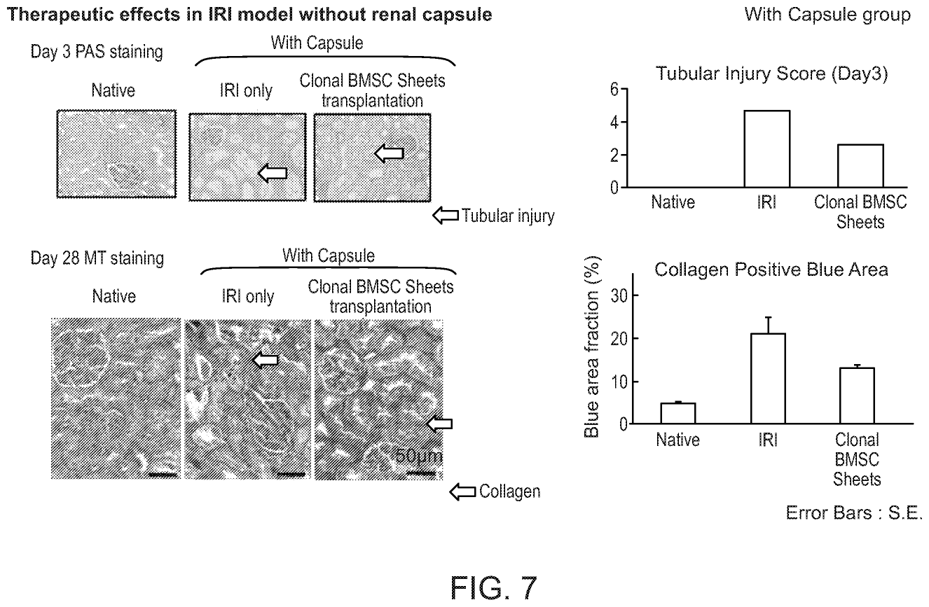

FIG. 7 shows BMSC sheet transplantation with kidney capsule. The upper row of FIG. 7 shows representative PAS staining images and tubular injury scores of native, IRI, and with capsule clonal BMSC sheets transplantation groups at 3-days after IRI. Tubular injury is indicated by black arrows in the IRI only and clonal MSC sheet groups. The clonal BMSC sheet suppresses the early phase of IRI injury as indicated by the clonal BMSC sheet group's lower tubular injury scores compared to the IRI group. The lower row shows representative MT staining images and the graph of collagen positive blue area fraction, indicating fibrotic areas, of Native, IRI, with capsule non-clonal BMSC sheet (WB), and with capsule clonal BMSC sheet transplantation groups at 28-days after IRI. The area of MT staining indicates collagen deposition (arrow). Clonal BMSC sheet transplantation with capsule shows the highest ability to inhibit fibrosis compared to the IRI and non-clonal BMSC sheet (WB) groups, as indicated by the lowest collagen staining.

### Example 3. Clonal BMSC Sheet Fabrication with Different Culture Conditions (Density and Culture Time) and Evaluation of Cytokine Production

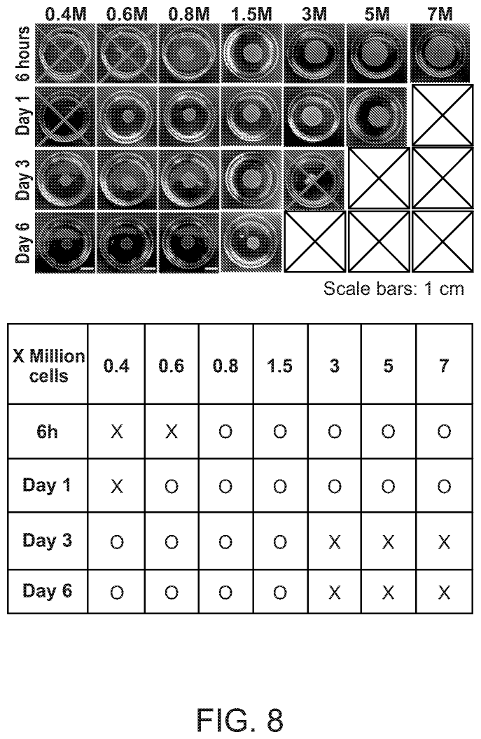

Clonal BMSCs were expanded from passage 8 to 10 on a tissue culture dish. Passage 10 cells were seeded at the density of 0.4-7 million cells/35 mm diameter temperature responsive cell culture dish (TRCD, CellSeed Inc., Tokyo, Japan) in basic media with ascorbic acid (50 ng/mL). Cells were detached by changing culture temperature from 37° C. to room temperature (RT) at 6 hours, 1, 3, or 6 days after seeding. As shown in FIG. 8, clonal BMSC sheets were detached as a sheet for the initial cell density of 0.8 million cells/35 mm TRCD at 6 hours, 0.6 million cells/35 mm TRCD at 1 day, and 0.4 million cells/35 mm TRCD at 3 and 6 days after seeding.

Clonal BMSC sheets were collected after detachment from TRCD at RT. Total RNA from cell sheets was extracted using trizol and PureLink RNA Mini Kt (Life Technologies, Carlsbad, USA) according to manufacturer's protocols. cDNA was prepared from 1 μg of total RNA using high capacity cDNA reverse transcription kits (Life Technologies). RT-PCR analysis was performed with TapMan Universal PCR Master Mix using an Applied Biosystems Step One instrument (Applied Biosystems™, Foster City, USA). Gene expression levels were assessed for the following genes: 1) glyceraldehyde 3-phosphate dehydrogenase (GAPDH, Hs99999905_m1) as a housekeeping gene, 2) HGF (Hs0037914_m1), 3) IL10 (Hs00961622_m1), and 4) VEGF (Hs99999070_m1). All primers were manufactured by Applied Biosystems. Relative gene expression levels were quantified by the comparative CT method. Gene expression levels were normalized to GAPDH expression levels. Gene expression levels are relative to levels of the single cell suspension group (SC).

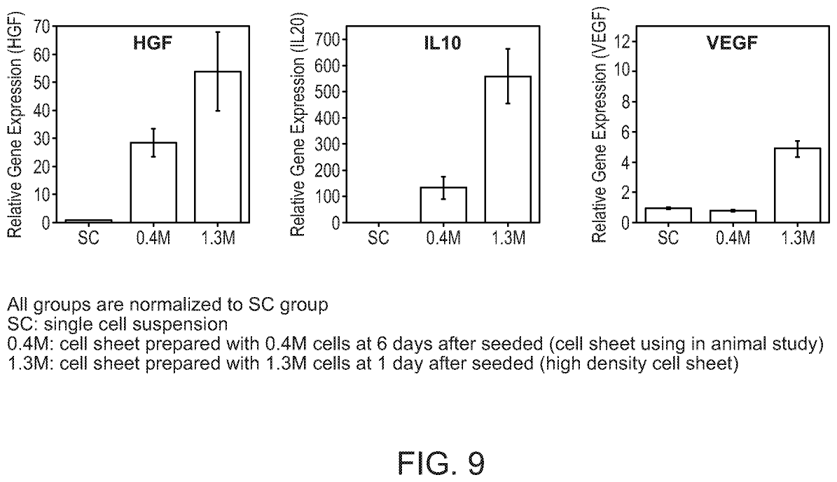

Gene expression levels of tissue regenerative cytokines (HGF, IL10, VEGF) of clonal BMSC sheets were compared with gene expression levels of single clonal BMSC suspensions (SC). As shown in FIG. 9, clonal BMSC sheets showed higher gene expression levels for HGF, IL10, and VEGF cytokine secretion, compared to single cell suspensions of clonal BMSCs (SC). Furthermore clonal BMSC sheets prepared with the density of 1.3 million cells/35 mm TRCD expressed higher gene expression levels (HGF, IL10, VEGF), compared to gene expression levels of clonal BMSC sheets prepared with the initial cell density of 0.4 million cells/35 mm TRCD.

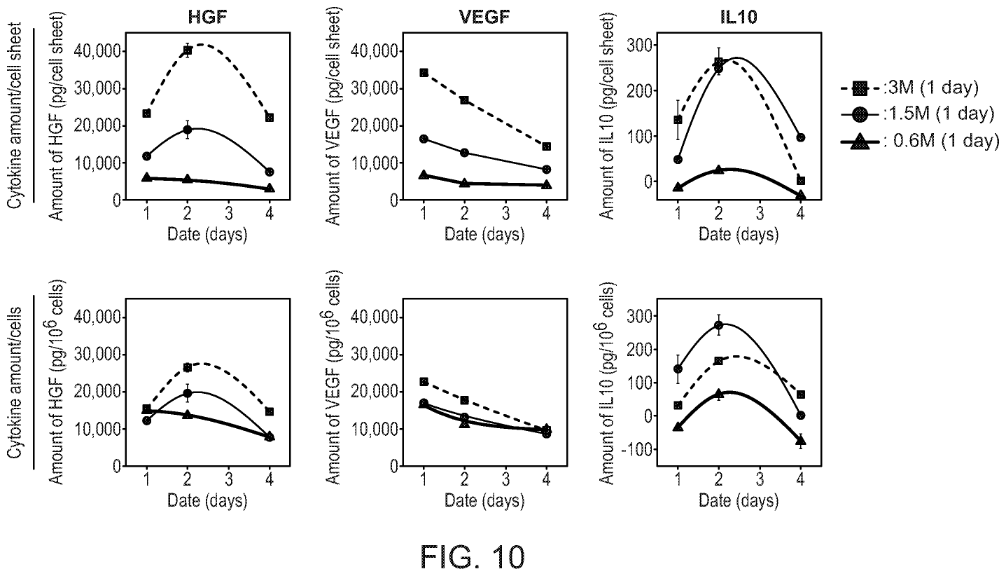

Clonal BMSC sheet media were exchanged with fresh media. Samples were cultured for 24 hours (37° C., 5.0% CO₂). The 24-hour supernatants (n=3 per group) were centrifuged at 1200 RPM for 5 min to collect soluble cytokines in the supernatant without cellular debris. The concentration of soluble HGF, VEG, IL10 secreted per clonal BMSC sheet was quantified using a human HGF, VEGF, IL10 Quantikine ELISA kit (R&D Systems, MN, USA). The cytokine amounts were normalized to the number of cell sheets or cells to determine cytokine amounts secreted per cell sheet or cell. Cytokine amounts secreted from clonal BMSC sheets with different initial cell density (0.6, 1.5, 3 million cells/35 mm TRCD) were detected. As shown in FIG. 10, higher initial cell density groups (1.5 and 3 million cells/35 mm TRCD) secreted higher amount of cytokines per cell sheet and cell, compared to 0.6 million cells/35 mm TRCD group.

### Example 4. Evaluation of Frozen BMSCs and Preparation of Cell Sheets

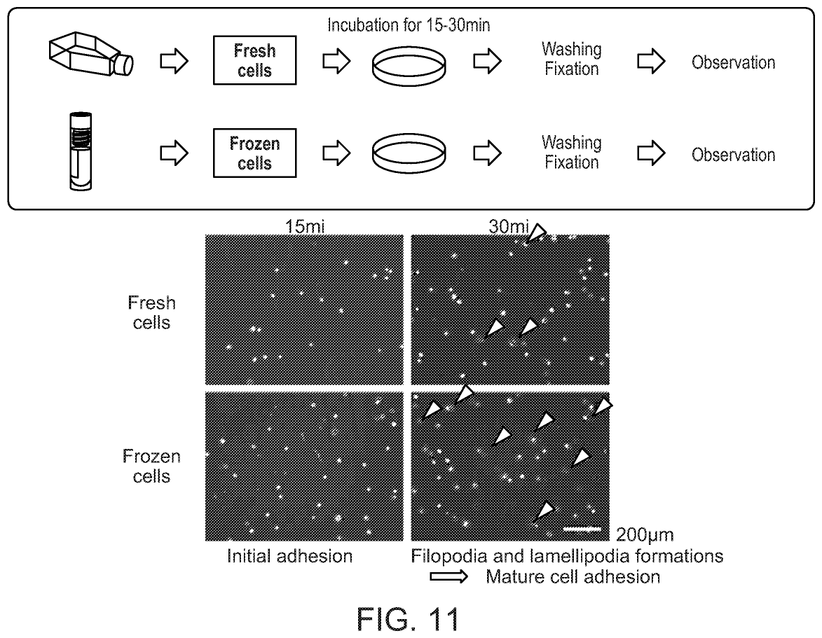

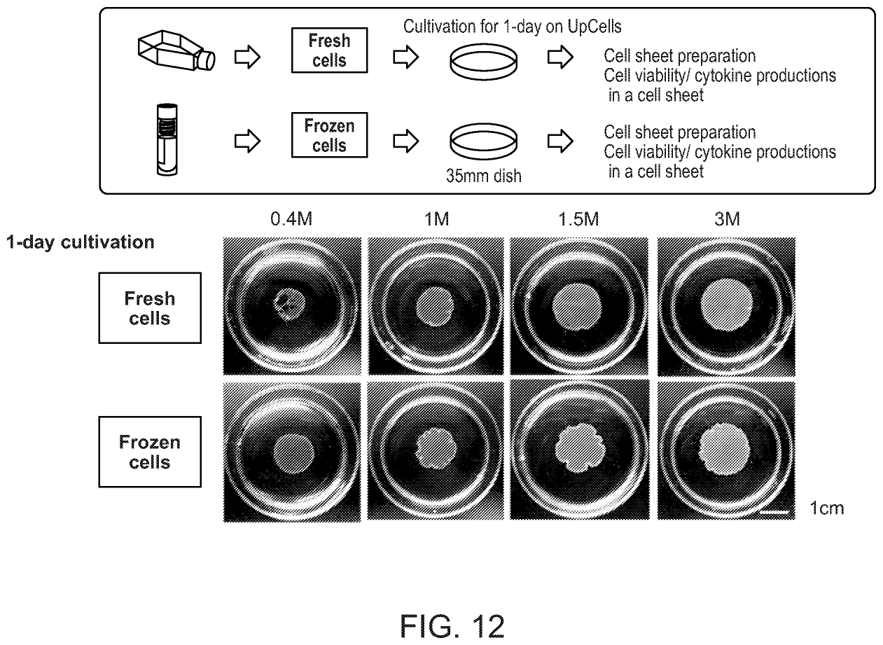

Fresh clonal BMSCs (fresh cells) were obtained from a cell culture surface by trypsin treatment at passage 9 (P9). Frozen clonal BMSCs (frozen cells) were prepared by freezing the banked P9 clonal BMSCs in liquid nitrogen tank. Both fresh and frozen cells were counted and seeded onto 35-mm cell culture dishes at the concentration of 5000 cells/cm² as passage 10 cells. After 15 or 30 minutes of incubation, non-adherent cells were washed out using PBS(−) and only adherent cells were observed on phase contrast microscope. As shown in FIG. 11, frozen cells showed high initial and mature cell adhesion ability compared to fresh cells. (Scale bar; 200 μm) Both fresh and frozen cells were seeded in cell culture dishes at 0.4, 1, 1.5, and 3 million (0.4M, 1.0M, 1.5M, and 3.0M) cells. Cell sheets prepared from fresh or frozen cells were harvested after 1-day cultivation. As shown in FIG. 12, the higher initial cell densities resulted in larger cell sheets. Frozen cells allowed preparation of cell sheets at a lower initial cell density compared to fresh cells. (Scale bar; 1 cm).

The cell sheets prepared from frozen cells were dissociated by collagenase and trypsin treatments, and the cell viabilities were determined by trypan blue staining. The viability of cells from sheets prepared from frozen cells or fresh cells are shown in Table 1 below. The cells from sheets prepared from frozen cells showed high cell viability, as did cells from sheets prepared from fresh cells.

TABLE 1
Viability of cells from sheets prepared
from frozen cells or fresh cells.

| Initial cell density | | |
|---|---|---|
| (cells per culture dish) | Cell Type | Cell Viability |
| 0.4M | Frozen | 97.8% |
| 1.0M | Frozen | 97.8% |
| 1.5M | Frozen | 97.8% |
| 3.0M | Frozen | 96.2% |
| 0.4M | Fresh | 96.1% |

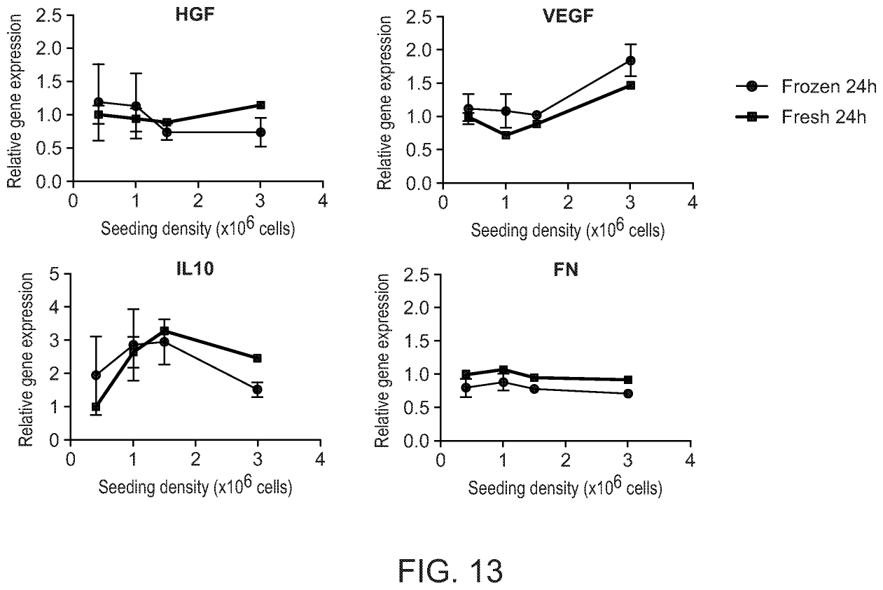

Fresh and frozen cell sheets were prepared at the initial cell density of 0.4, 1, 1.5, and 3 million (0.4M, 1.0M, 1.5M, and 3.0M) and harvested after 1-day cultivation. Prepared cell sheets were employed for qPCR to investigate their cytokine production. As shown in FIG. 13, sheets prepared from frozen cells at each initial cell density showed comparable cytokine production to sheets prepared from fresh cells, suggesting that frozen cells can be an option for cell sheet production. In particular, frozen cell stocks are beneficial to perform rapid cell sheet production based on their high initial cell adhesion ability in culture.

### Example 5

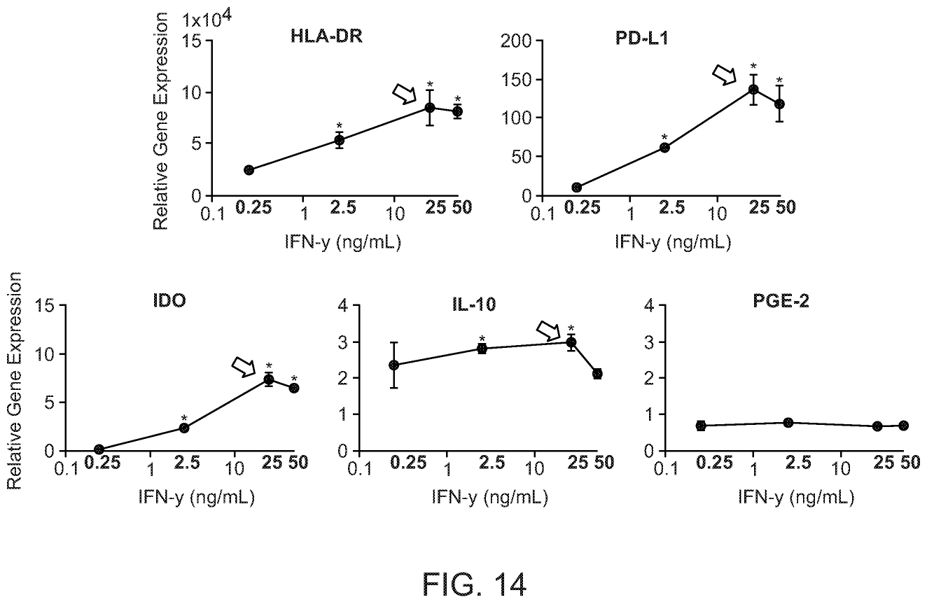

Clonal bone marrow-derived mesenchymal stem cell (BMSC) sheets were fabricated at passage 10 by seeding 0.4×10⁶ cells per 35 mm TRCD. At day 6, cell sheets were detached and replated onto a 1 μm pore insert well and cultured with media containing either 0, 0.25, 2.5, 25, or 50 ng/mL IFN-γ. After 2 days, cell sheets were collected from the insert well and processed for qRT-PCR analysis. As shown in FIG. 14, clonal BMSC sheets upregulate expression of the immunomodulatory molecules HLA-DR, PD-L1, IDO, IL-10 and PGE-2 in response to interferon gamma (IFN-γ). Clonal BMSC sheets exhibited peak upregulation of immunomodulatory genes when exposed to 25 ng/mL IFN-γ. Gene expression was represented as fold change normalized to a non-primed control cell sheet under the same conditions. (Sample number; n=2).

### Example 6

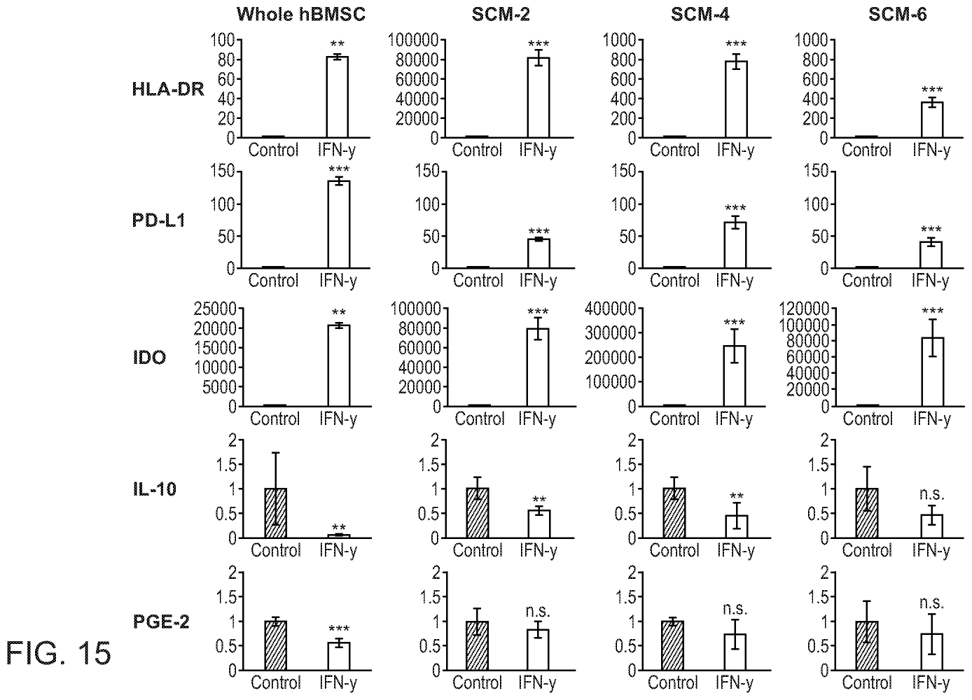

Clonal and whole BMSC sheets were fabricated at passage 10 by seeding 0.4×10⁶ cells per 35 mm TRCD. At day 4 of cell sheet culture, 25 ng/mL IFN-γ was added to the culture to initiate 2-days of priming before detachment. At day 6 of cell sheet culture, cell sheets were detached and processed for qRT-PCR analysis. As shown in FIG. 15, cell sheets fabricated with IFN-γ supplementation exhibit upregulation of immunomodulatory molecules. Prior to cell sheet detachment, 25 ng/mL IFN-γ was added at 2 days. Clonal BMSC sheets primed for 2-days prior to detachment exhibited significant increase in gene expressions of HLA-DR, PD-L1, and IDO. Gene expressions were represented as fold change normalized to respective non-primed control cell sheet fabricated under the same conditions. (Sample number; n=6).

Conclusion: 48 hr IFN-7 primed hBMSC sheets exhibited upregulated IDO and HLA-DR, stable PGE-2, and downregulated IL-10 immediately following cell sheet detachment. IFN-γ priming had the greatest effect on IDO gene expression. Since, IDO gene expression is associated with T cell inhibition, primed cell sheets may be useful in reducing inflammation.

### Example 7

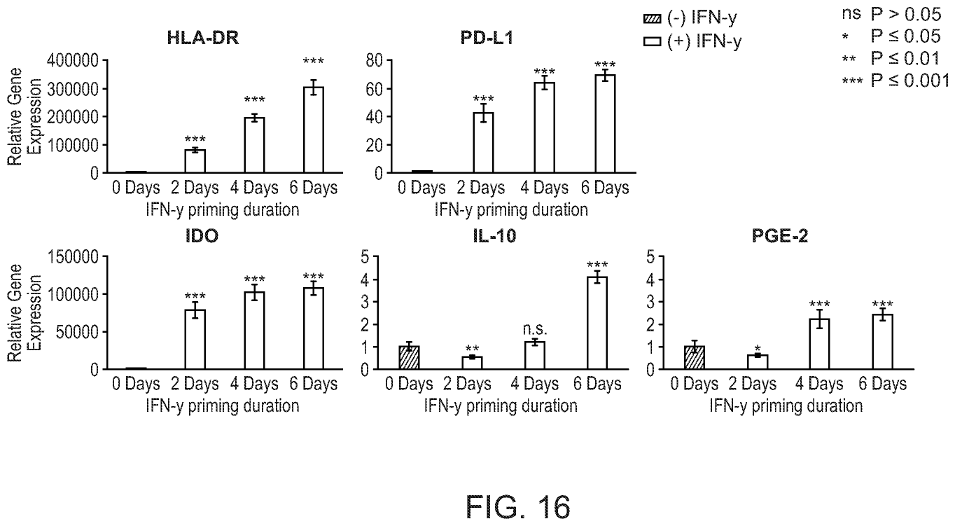

Clonal BMSC sheets were fabricated at passage 10 by seeding 0.4×10⁶ cells per 35 mm TRCD. 25 ng/mL of IFN-γ was added to culture either at day 0 (time of seeding), day 2, or day 4. At day 6 of cell sheet culture, cell sheets were detached and processed for qRT-PCR. As shown in FIG. 16, increased duration of IFN-γ priming was associated with further increased gene expression of immunomodulatory molecules. Clonal BMSC sheets fabricated after 4-days and 6-days of IFN-γ priming exhibited upregulation of immunomodulatory genes (HLA-DR, PD-L1, IDO, IL-10, and PGE-2) with highest expression after 6-days of priming. Gene expression was represented as fold change normalized to respective non-primed control cell sheet fabricated under the same conditions. (Sample number; n=6).

### Example 8

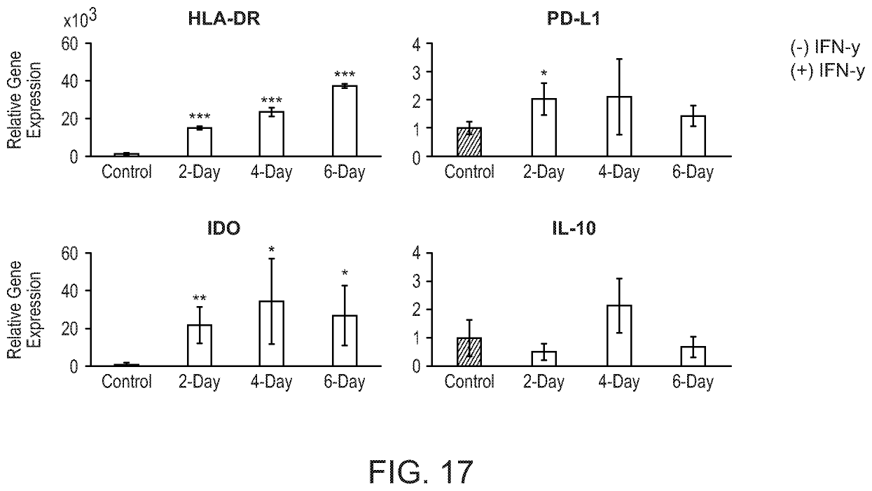

Clonal BMSC sheets were fabricated at passage 10 by seeding 0.4×10⁶ cells per 35 mm TRCD. 25 ng/mL of IFN-γ was added to culture either at day 0 (time of seeding), day 2, or day 4. At day 6 of cell sheet culture, cell sheets were detached replated onto a 1 μm pore insert well and cultured with standard culture media. After 4 days, cell sheets were collected from the insert well and processed for qRT-PCR analysis. Cell sheets fabricated with either 6-days, 4-days, 2-days, or 0-days IFN-γ supplementation were replated in normal culture conditions for 4-days to analyze stability of IFN-γ effect. The results indicated that even after removal of IFN-γ clonal BMSCs continue to exhibit upregulated gene expression of HLA-DR, PD-L1, IDO, and IL-10. See FIG. 17. Clonal cell sheets fabricated with 4-days of IFN-γ priming exhibited highest gene expression of immunosuppressive (IDO, PD-L1, and IL-10) molecules. Gene expression was represented as fold change normalized to respective non-primed control cell sheet fabricated under the same conditions. (Sample number; n=4).

### Example 9

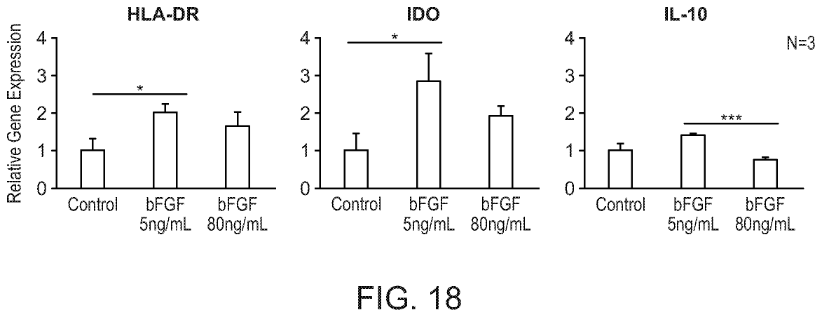

Clonal BMSC sheets were fabricated at passage 10 by seeding 0.4×10⁶ cells per 35 mm TRCD. At day 4 of cell sheet culture, either 5 or 80 ng/mL of bFGF was added to the culture to initiate 2-days of priming before detachment. At day 6 of cell sheet culture, cell sheets were detached and processed for qRT-PCR analysis. As shown in FIG. 18, cell sheets fabricated with bFGF supplementation exhibit upregulation of immunomodulatory molecules. For example, clonal BMSC sheets primed for 2-days prior to detachment exhibited a significant increase in HLA-DR, IDO, and IL-10. Gene expression was represented as fold change normalized to respective non-primed control cell sheet fabricated under the same conditions. (Sample number; n=3)

1. A human bone marrow-derived mesenchymal stem cell sheet comprising one or more layers of human bone marrow-derived mesenchymal stem cells (hBMSCs), wherein the cell sheet is prepared from a human clonal bone marrow-derived mesenchymal stem cell line generated from a single cell.

2. The cell sheet of claim 1, wherein the hBMSCs in the cell sheet are confluent.

3. The cell sheet of claim 1 or 2, wherein the human clonal bone marrow-derived mesenchymal stem cell line has been frozen before preparation of the cell sheet.

4. The cell sheet of claim 1 or 2, wherein the human clonal bone marrow-derived mesenchymal stem cell line has not been frozen before preparation of the cell sheet.

5. The cell sheet of any one of claims 1 to 4, wherein the cell sheet consists essentially of hBMSCs.

6. The cell sheet of any one of claims 1 to 4, wherein at least 90% of cells in the cell sheet are hBMSCs.

7. The cell sheet of any one of claims 1 to 6, wherein the hBMSCs in the cell sheet express one or more cytokines selected from the group consisting of human growth factor (HGF), vascular endothelial growth factor (VEGF), Fibroblast Growth Factor 2 (FGF2), interleukin-10 (IL-10), Indoleamine 2,3-dioxygenase (IDO), and Prostaglandin E2 (PGE2).

8. The cell sheet of claim 7, wherein expression of the one or more cytokines in the cell sheet is increased relative to a suspension of hBMSCs containing an equivalent number of cells.

9. The cell sheet of any one of claims 1 to 8, wherein initial seeded cell density of the human clonal bone marrow-derived mesenchymal stem cell line in a cell culture support used to prepare the cell sheet is from 4.5×10⁴ to 3.4×10⁵ cells/cm².

10. A composition comprising the cell sheet of any one of claims 1 to 9 and a polymer-coated culture support that is removable from the cell sheet.

11. A method for producing a human bone marrow-derived mesenchymal stem cell sheet comprising one or more layers of confluent human bone marrow-derived mesenchymal stem cells (hBMSCs), the method comprising:
a) culturing hBMSCs in culture solution on a temperature-responsive polymer which has been coated onto a substrate surface of a cell culture support, wherein the hBMSCs in culture solution are a human clonal bone marrow-derived mesenchymal stem cell line generated from a single cell, and wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

12. The method of claim 11, wherein the hBMSCs in culture solution have been frozen before the culturing step (a).

13. The method of claim 11, wherein the hBMSCs in culture solution have not been frozen before the culturing step (a).

14. The method of any one of claims 11 to 13, wherein the culturing step (a) comprises adding hBMSCs to the culture solution at an initial cell seeding density from 4.5×10⁴ to 3.4×10⁵ cells/cm².

15. The method of any one of claims 11 to 14, further comprising culturing the hBMSCs through multiple subcultures prior to the culturing step (a).

16. The method of claim 15, wherein 2 to 10 subcultures of the hBMSCs are performed prior to the culturing step (a).

17. The method of any one of claims 11 to 16, wherein the hBMSCs are cultured in the culture solution on the temperature-responsive polymer for at least 1 day before the adjusting step (b).

18. The method of any one of claims 11 to 16, wherein the hBMSCs are cultured in the culture solution on the temperature-responsive polymer for 1 to 3 days.

19. The method of any one of claims 11 to 18, wherein the adjusting step (b) is performed when the hBMSCs in culture solution are confluent.

20. A cell sheet produced by the method of any one of claims 11 to 19.

21. A method of transplanting a cell sheet to a subject comprising applying the cell sheet of any one of claims 1 to 10 or 20 to a tissue of a subject.

22. The method of claim 21, wherein the tissue is kidney tissue.

23. The method of claim 21 or 22, wherein the subject has received a kidney transplant.

24. The method of claim 21 or 22, wherein the subject has an acute kidney injury.

25. The method of claim 21 or 22, wherein the subject has kidney tubule injury.

26. The method of any one of claims 22 to 25, wherein applying the cell sheet to the kidney tissue results in migration of cells from the cell sheet into parenchyma tissue of the kidney.

27. The method of any one of claims 22 to 26, wherein applying the cell sheet to the kidney tissue results in increased levels in the kidney of one or more cytokines selected from the group consisting of human growth factor (HGF), vascular endothelial growth factor (VEGF), Fibroblast Growth Factor 2 (FGF2), interleukin-10 (IL-10), Indoleamine 2,3-dioxygenase (IDO), and Prostaglandin E2 (PGE2), relative to a kidney that is not contacted with the cell sheet.

28. A method of suppressing renal fibrosis in a subject comprising applying the cell sheet of any one of claims 1 to 10 or 20 to kidney tissue of a subject, thereby suppressing renal fibrosis in the subject.

29. The method of claim 28, wherein applying the cell sheet to the kidney tissue suppresses renal fibrosis to a greater extent than a hBMSC sheet prepared from hBMSCs that are not a clonal cell line.

30. The method of any one of claims 21 to 29, wherein the hBMSCs in the cell sheet are allogeneic to the subject.

31. The method of any one of claims 21 to 30, wherein the subject is human.

32. A bone marrow-derived mesenchymal stem cell sheet comprising one or more layers of bone marrow-derived mesenchymal stem cells (hBMSCs), wherein the cell sheet is prepared from a human clonal bone marrow-derived mesenchymal stem cell line generated from a single cell, and wherein the cell sheet is treated with interferon gamma (IFN-γ) or basic fibroblast growth factor (bFGF).

33. The cell sheet of claim 32, wherein the hBMSCs in the cell sheet are confluent.

34. The cell sheet of claim 32 or 33, wherein the cell sheet consists essentially of hBMSCs.

35. The cell sheet of any one of claims 32 to 34, wherein at least 90% of cells in the cell sheet are hBMSCs.

36. The cell sheet of any one of claims 32 to 35, wherein the hBMSCs in the cell sheet exhibit increased expression of one or more proteins selected from the group consisting of HLA-DR, PD-L1, Indoleamine 2,3-dioxygenase (IDO), interleukin 10 (IL-10) and Prostaglandin E2 (PGE-2) relative to an hBMSC sheet that is not treated with IFN-γ or bFGF.

37. A composition comprising the cell sheet of any one of claims 32 to 26 and a polymer-coated culture support that is removable from the cell sheet.

38. A method for producing a human bone marrow-derived mesenchymal stem cell (hBMSC) sheet comprising one or more layers of confluent human bone marrow-derived mesenchymal stem cells (hBMSCs), the method comprising:
a) culturing hBMSCs in culture solution on a temperature-responsive polymer which has been coated onto a substrate surface of a cell culture support, wherein the hBMSCs in culture solution are a human clonal bone marrow-derived mesenchymal stem cell line generated from a single cell, and wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened;
c) detaching the cell sheet from the culture support; and
d) culturing the cell sheet in culture solution containing interferon gamma (IFN-γ) or basic fibroblast growth factor (bFGF).

39. The method of claim 38, wherein the culturing step (a) comprises adding hBMSCs to the culture solution at an initial cell seeding density from 4.5×10⁴ to 3.4×10⁵ cells/cm².

40. The method of claim 38 or 39, further comprising culturing the hBMSCs through multiple subcultures prior to the culturing step (a).

41. The method of claim 40, wherein 2 to 10 subcultures of the hBMSCs are performed prior to the culturing step (a).

42. The method of any one of claims 38 to 41, wherein the hBMSCs are cultured in the culture solution on the temperature-responsive polymer for at least 1 day before the adjusting step (b).

43. The method of any one of claims 38 to 42, wherein the hBMSCs are cultured in the culture solution on the temperature-responsive polymer for 1 to 3 days.

44. The method of any one of claims 38 to 43, wherein the adjusting step (b) is performed when the hBMSCs in culture solution are confluent.

45. A cell sheet produced by the method of any one of claims 38 to 44.

46. A method of transplanting a cell sheet to a subject comprising applying the cell sheet of any one of claims 32 to 41 or 45 to a tissue of a subject.

47. A method of modulating immune response in a subject comprising applying the cell sheet of any one of claims 32 to 41 or 45 to a tissue of a subject.

48. The method of claim 46 or 47, wherein applying the cell sheet to the tissue results in increased levels in the tissue of one or more proteins selected from the group consisting of HLA-DR, PD-L1, Indoleamine 2,3-dioxygenase (IDO), interleukin 10 (IL-10) and Prostaglandin E2 (PGE-2), relative to a tissue that is not contacted with the cell sheet.

49. The method of any one of claims 46 to 48, wherein the hBMSCs in the cell sheet are allogeneic to the subject.

50. The method of any one of claims 46 to 49, wherein the subject is human.
